(19) United States
(12) Patent Application Publication
(10) Pub. No.: US 2016/0058754 A1
(43) Pub. Date: Mar. 3, 2016

(54) Nalmefene for Treatment of Patients with Anxiety Disorder

(71) Applicant: H. LUNDBECK A/S, Valby (DK)

(72) Inventors: Didier Meulien, Boulogne Billancourt (FR); David Gruhn, Valby (DK); Lars Torup, Værløse (DK); Björn Steiniger-Brach, Brønshøj (DK)

(73) Assignee: H. Lundbeck A/S, Valby (DK)

(21) Appl. No.: 14/784,737

(22) PCT Filed: Apr. 16, 2014
(86) PCT No.: PCT/EP2014/057679

§ 371 Date: Oct. 15, 2015

(30) Foreign Application Priority Data
Apr. 17, 2013 (DK) PA201300229

(51) Int. Cl. A61K 31/485; A61K 45/06

(52) U.S. Cl. CPC A61K 31/485; A61K 45/06

(57) ABSTRACT

The present invention relates to nalmefene for use in the treatment of anxiety disorders. The present invention further relates to nalmefene for use in the treatment of patients with alcohol dependence who have a co-morbid anxiety disorder. The invention further relates to nalmefene for use in the treatment of an anxiety disorder in said patients.

### FIELD OF THE INVENTION

The present invention relates to nalmefene for use in the treatment of anxiety disorders. The present invention further relates to nalmefene for use in the treatment of patients with alcohol dependence who have a co-morbid anxiety disorder. The invention further relates to nalmefene for use in the treatment of an anxiety disorder in said patients.

### BACKGROUND OF THE INVENTION

Nalmefene [17-(cyclopropylmethyl)-4,5-alpha-epoxy-6-methylenemorphinan-3,14-diol] has the following general formula:

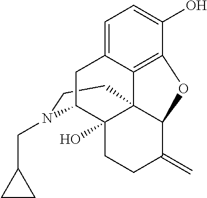

and can be prepared using methods that are well known in the art e.g. starting by manufacturing of naltrexone from noroxymorphone as described in WO 2012/059103 and subsequently manufacturing nalmefene from naltrexone e.g. by the Wittig reaction as described in WO 2010/136039.

Nalmefene is an opioid system modulator with a distinct μ, δ, and κ receptor profile. In vitro studies have demonstrated that nalmefene is a selective opioid receptor ligand with antagonist activity at the μ and δ receptors and partial agonist activity at the κ receptor. Acute alcohol intake was shown to result in mesolimbic dopamine release (facilitated by the release of β-endorphins), which can provide positive reinforcement. Nalmefene is thought to counteract the reinforcement effects and to reduce alcohol consumption, possibly by modulating these cortico-mesolimbic functions.

The efficacy and tolerability of nalmefene in the treatment of alcohol dependence have been evaluated in three phase III studies (two confirmatory 6-month efficacy studies and one 1-year safety study) conducted by Lundbeck (Mann et al. Extending the Treatment Options in Alcohol Dependence: A Randomized Controlled Study of As-Needed. Nalmefene. Biol Psychiatry, (2013); 73: 703-713; Gual et al. A randomised, double-blind, placebo-controlled, efficacy study of nalmefene, as-needed use, in patients with alcohol dependence. European Neuropsychopharmacology, (2013); 23(11): 1432-1442; van den Brink et al., Long-term efficacy, tolerability and safety of nalmefene as-needed in patients with alcohol dependence: A 1-year, randomised controlled study. J. Psychopharmacol., published online before print Mar. 26, 2014, doi: 10.1177/0269881114527362) and 5 studies in alcohol use disorders conducted by the company Biotie (Karhuvaara et al. Alcohol. Clin Exp Res. (2007); 31: 1179-1187).

A marketing authorisation has recently been granted (February 2013) for oral nalmefene in the European Union (EU) under the tradename Selincro® for the reduction of alcohol consumption in adult patients with alcohol dependence.

Co-occurrence of alcohol dependence and anxiety disorders are common and primarily based on findings from epidemiological studies which illustrate the complexity of the comorbidity between alcohol dependence on one side and anxiety disorders on the other side (Grant and Hartford, Drug and Alcohol Dependence, (1995), Vol. 39: 197-206.; Swendsen et al., Comprehensive Psychiatry, (1998), Vol. 38(4): 176-184; Kessler et al., Arch. Gen. Psychiatry, (1997), Vol. 54: 313-321).

These studies with often very large samples have shown that there is a high level of lifetime comorbidity between depressive and anxiety disorders. Patients with anxiety disorders have an increased risk of suffering from alcohol dependence compared to patients without anxiety disorders. Likewise, patients with alcohol dependence have an increased risk of comorbid anxiety disorders compared to patients without alcohol dependence. Subjects with an anxiety disorder have approximately double the odds of also having alcohol dependence (Regier, et al. JAMA (1990), 264: 2511-2518. Kessler, et al. Archives of General Psychiatry (1994), 51: 8-19. Merikangas et al. Addictive Behaviors (1998), 23: 893-907).

Table 1 presents the lifetime co-occurrence of alcohol dependence in anxiety disorders (lifetime diagnosis) based on data from the National Comorbidity Study (Kessler et al., Arch. Gen. Psychiatry, (1997), Vol. 54: 313-321). In patients with alcohol dependence the lifetime prevalence of any anxiety disorders is high: 35.8% in men and up to 60.7% in women and variation among specific conditions is observed. Furthermore, there is a possibility of suffering from more than one comorbid condition.

TABLE 1
Lifetime Co-occurrence of Alcohol Dependence with Other Lifetime
National Comorbidity Survey/DSM-III-R Disorders, by Sex.

| Lifetime | | | | | | |
|---|---|---|---|---|---|---|
| Disorders | Men (N = 806) | | | Women (N = 336) | | |
| Anxiey | %^{a} | OR^{b} | 95% CI | %^{a} | OR^{b} | 95% CI |
| GAD | 8.6 | 3.86 | [2.27; 6.58] | 15.7 | 3.01 | [1.70; 5.32] |
| Panic | 3.6 | 2.27 | [1.11; 4.64] | 12.0 | 2.98 | [1.74; 5.09] |
| Agoraphobia | 6.5 | 1.82 | [1.04; 3.20] | 18.5 | 2.53 | [1.69; 3.80] |
| Social phobia | 19.3 | 2.41 | [1.76; 3.31] | 30.3 | 2.62 | [2.00; 2.43] |
| Simple phobia | 13.9 | 3.11 | [2.29; 4.22] | 30.7 | 2.63 | [1.84; 3.77] |
| PTSD | 10.3 | 3.20 | [1.86; 5.48] | 26.2 | 3.60 | [2.56; 5.05] |
| Any | 35.8 | 2.22 | [1.67; 2.95] | 60.7 | 3.08 | [2.20; 4.30] |

^{a}prevalences of the disorders represented in the rows among respondents with a lifetime diagnosis of alcohol dependence
^{b}all ORs are significant at the 0.05 level, 2-tailed test

Comorbidity between alcohol dependence and anxiety disorder is also underlined by the various temporal patterns for the co-occurrence of these conditions, illustrated by the order of their onset such as primary, secondary or simultaneous onset). Table 2 (Swendsen et al., Comprehensive Psychiatry, (1998), Vol. 38(4): 176-184) shows that onset of anxiety disorder in relation to alcohol dependence can vary. For panic disorder an onset before alcohol dependence is as frequent as a later onset. The main exception are phobias which most often have their onset in childhood, adolescence or early adulthood and therefore as a rule before the onset of an alcohol dependence.

TABLE 2
Retrospective Estimates for Order of Onset of Alcohol
Abuse/Dependence With Anxiety Disorders.

| | Phobias^{#} | | | Panic | | |
|---|---|---|---|---|---|---|
| Age of | | | Puerto | | | Puerto |
| Onset | ECA | NCS | Rico | ECA | NCS | Rico |
| A < B | 13.1% | 19.0% | 27.3% | 45.2% | 62.3% | 33.3% |
| A = B | 3.2% | 4.2% | 9.1% | 10.2% | 5.1% | 16.7% |
| A > B | 83.0% | 76.7% | 63.6% | 44.5% | 32.7% | 50.0% |
| Totalno. | 315 | 585 | 33 | 23 | 96 | 6 |

Abbreviations: A: alcoholism, B: index disorder
^{#}Any agoraphobia, simple phobia, or social phobia.

Anxiety disorders and alcohol dependence carry a significant risk for the development of the other. And also the severity in one disorder is associated with severity in the other. The presence of anxiety disorders has been reported to have an impact on the severity of alcohol dependence. On the other hand, the presence of alcohol dependence is associated with greater increases in the severity of depression or anxiety, indicated by a higher number of symptoms (Swendsen and Merikangas, Clin. Psychol. Rev., (2000); Vol. 20(2):173-189).

As described above the co-occurrence is quite common and this advocates for concurrent treatment of the alcohol use disorder and anxiety disorders because each is likely to perpetuate the other. Current expert consensus supports concurrent psychosocial and psychopharmacological treatment of comorbid anxiety and substance use disorders (Watkins et al. Psychiatr Serv. (2005), 56: 913-926).

However, there is some limitation in the use of anxiolytics in patients with alcohol dependence. The use of benzodiazepines in an alcohol dependent population is controversial (excluding use for alcohol withdrawal) and should not be undertaken without expert advice and monitoring. Subjects with alcohol dependence may be at higher risk of benzodiazepine misuse and dependence because of their greater rewarding effects (Ciraulo & Nace. American Journal of Addiction. (2000), 9: 276-284.)

The impact of SSRIs in treating comorbid alcohol and anxiety is not clear. When considering the use of SSRIs, it is important to recognise that treating alcoholism either with or without a comorbid depressive disorder, SSRIs may not only result in no improvement but may actually reduce the impact of psychological treatment such as cognitive behavioural therapy (CBT) (Kranzler et al, Alcohol. Clin. Exp. Res. (1996), 20(9): 1534-1541; Lingford-Hughes et al., J. Psychopharmacol. (2012), Vol. 26(7): 899-952) as well further impact on the sleep disturbances frequently reported in such population. Thus, it should be borne in mind that SSRIs are not necessarily the drug of choice in patients with comorbid alcohol use disorder and anxiety disorder.

Therefore there is a need for new treatments for use in patients with alcohol dependence who have a co-morbid anxiety disorder. In particular, there is a need for new treatments which could give rise to advantages such as e.g. improved efficacy and/or a different side effect profile compared to existing treatments.

### SUMMARY OF THE INVENTION

The present invention relates to nalmefene for use in the treatment of an anxiety disorder.

In one embodiment, the invention relates to nalmefene for use in the treatment of a patient with alcohol dependence who has a co-morbid anxiety disorder.

In one embodiment, the invention relates to a pharmaceutical composition comprising nalmefene and a second compound, which an antianxiety agent, and optionally acceptable carriers or diluents.

In one embodiment, the invention relates to a kit comprising nalmefene together with a second compound, which an antianxiety agent.

In one embodiment, the invention relates to a method for the treatment of an anxiety disorder, which method comprises administering a pharmaceutically acceptable amount of nalmefene to a patient in need thereof.

In one embodiment, the invention relates to a method for reduction of alcohol consumption and for the treatment of an anxiety disorder, which method comprises administering a pharmaceutically acceptable amount of nalmefene to a patient in need thereof.

### BRIEF DESCRIPTION OF DRAWINGS

For all figures, -□-=placebo (PBO), -▪-=nalmefene (NMF), “B” denotes baseline. Number of patients “N” for placebo (PBO) and nalmefene (NMF), respectively throughout the study is indicated at the X-axis. Patients with and without an anxiety disorder at baseline were classified according to their ongoing medical history coded by the Medical Dictionary for Regulatory Activities (MedDRA).

FIGS. 1-2 show the change from baseline in monthly Heavy Drinking days (HDDs) and Total Alcohol Consumption (TAC) (g/day) in patients with an anxiety disorder at baseline vs. patients without an anxiety disorder at baseline.

FIGS. 1a-1b show the change from baseline in monthly HDDs. X-axis: time (months); Y-axis: change from baseline in mean HDD.

FIG. 1a: Patients without an anxiety disorder at baseline, change in monthly HDD.

FIG. 1b: Patients with an anxiety disorder at baseline, change in monthly HDD.

FIGS. 2a-2b show the change from baseline in monthly TAC (g/day). X-axis: time (months); Y-axis: change from baseline in mean TAC.

FIG. 2a: Patients without an anxiety disorder at baseline, change in monthly TAC.

FIG. 2b: Patients with an anxiety disorder at baseline, change in monthly TAC.

FIGS. 3-9 indicate change from baseline in POMS scores in patients with an anxiety disorder at baseline vs. patients without an anxiety disorder at baseline. X-axis: time (weeks); Y-axis: change from baseline in mean POMS.

FIG. 3a. Patients without an anxiety disorder at baseline, change in POMS total mood disturbance (TMD).

FIG. 3b: Patients with an anxiety disorder at baseline, change in POMS total mood disturbance (TMD).

FIG. 4a. Patients without an anxiety disorder at baseline, change in POMS Tension-Anxiety.

FIG. 4b: Patients with an anxiety disorder at baseline, change in POMS Tension-Anxiety.

FIG. 5a. Patients without an anxiety disorder at baseline, change in POMS Depression-Rejection.

FIG. 5b: Patients with an anxiety disorder at baseline, change in POMS Depression-Rejection.

FIG. 6a. Patients without an anxiety disorder at baseline, change in POMS Anger-Hostility.

FIG. 6b: Patients with an anxiety disorder at baseline, change in POMS Anger-Hostility.

FIG. 7a. Patients without an anxiety disorder at baseline, change in POMS Vigour.

FIG. 7b: Patients with an anxiety disorder at baseline, change in POMS Vigour.

FIG. 8a. Patients without an anxiety disorder at baseline, change in POMS Fatigue.

FIG. 8b: Patients with an anxiety disorder at baseline, change in POMS Fatigue.

FIG. 9a. Patients without an anxiety disorder at baseline, change in POMS Confusion.

FIG. 9b: Patients with an anxiety disorder at baseline, change in POMS Confusion.

### DEFINITIONS

Throughout the description, the term “nalmefene” is intended to include any form of the compound, such as the free base and pharmaceutically acceptable salts. The free base and pharmaceutically acceptable salts include anhydrous forms and solvated forms such as hydrates. The anhydrous forms and the solvates include amorphous and crystalline forms. In a particular embodiment, nalmefene is in the form of a hydrochloride salt. In a more particular embodiment, nalmefene is in the form of the hydrochloride dihydrate. Throughout the application, when a dose is specified for nalmefene, said dose is calculated as the free base, i.e. when the nalmefene dose is 18 mg this corresponds to 18 mg of nalmefene free base.

In the present context, the term “total alcohol consumption” abbreviated TAC indicates average total alcohol consumption measured in g/day

In the present context, the term “heavy drinking day” abbreviated HDD indicates a day with a total alcohol consumption ≧60 g for men and ≧40 g for women.

In the present context, “as-needed dosing” indicates that on each day a patient perceives a risk of drinking alcohol, one dose of nalmefene should be taken, preferably 1-2 hours prior to the anticipated time of drinking. If the patient has started drinking alcohol without taking nalmefene, the patient should take one tablet as soon as possible after that.

As used herein, the term “drinking risk level” abbreviated DRL is defined according to the criterias defined by the World Health Organization in “International Guide for Monitoring Alcohol Consumption and Related Harm” (2000), WHO, as outlined in Table 3 below.

TABLE 3
WHO Drinking Risk Levels (DRLs) of Alcohol Consumption

Total Alcohol Consumption (g/day)

| DRL | Men | Women |
|---|---|---|
| Very high risk | >100 | >60 |
| High risk | >60 to 100 | >40 to 60 |
| Medium risk | >40 to 60 | >20 to 40 |
| Low risk | 1 to 40 | 1 to 20 |

Drinking Risk Levels according to Table 3 can be assessed e.g. by calculating mean daily alcohol consumption in g/day over a period such as 1 week or longer, such as 2 weeks or longer, such as 3 weeks or longer, such as 4 weeks or longer, such as 1 month or longer such as 2 months or longer, such as 3 months or longer, such as 4 months or longer, such as 5 months or longer, such as 6 months or longer, such as about 1 year. Assessment of DRL can be performed by specialists and/or physicians such as general practitioners and/or other health care providers based on patients estimates of their alcohol consumption.

Throughout the application, the term “high risk” or “at least high risk” is intended to include the two groups defined as “high risk” and “very high risk” according to WHOs drinking risk levels listed in Table 3, i.e. patients having drinking risk level corresponding to a total alcohol consumption of >60 g/day of pure alcohol for men and >40 g/day for women. The present invention does not distinguish between patients with high and very high drinking risk levels, and when the terms “high drinking risk level” or “high DRL” are used in a claim or in an embodiment of the invention it is intended to include both the group defined as “high risk” and the group defined as “very high risk” according to WHOs drinking risk levels listed in Table 3.

As used herein, the terms “motivational support” and “counselling focused on enhanced treatment adherence and reduced alcohol consumption” indicate psychological motivation-enhancing interventions and can be used interchangeably with the terms “psychosocial support” or “psychosocial intervention focused on treatment adherence and reducing alcohol consumption”. Said motivational support can be administered by a specialist and/or a physician such as a general practitioner and/or other health care providers. One example of such interventions is the BRENDA model, which is a time-limited, patient-centered clinical motivational intervention that complements the use of medication with focus on changing behavior and increasing medication adherence. The BRENDA model has been described by Starosta et al., J. Psychiatr. Pract. (2006), Vol. 12(2): 80-89, the entire contents of which are incorporated herein by reference. The term “initial motivational support” indicates such motivation-enhancing interventions provided to the patient prior to treatment with nalmefene. The term “ongoing motivational support” indicates such motivation-enhancing interventions provided to the patient concurrent to treatment with nalmefene e.g. on a recurrent basis.

In the present context, “Pharmaceutical composition” refers to a dose form such as an oral dose form, such as a solid oral dose form, typically tablets or capsules. “Pharmaceutical compositions of the present invention” refers to all pharmaceutical compositions covered by the claims and description.

In the present context, a “unit dosage form” refers to a formulation unit of a pharmaceutical composition e.g. one tablet or capsule.

In the present context, “therapeutically effective amount” of a compound means the amount/dose of a compound or pharmaceutical composition that is sufficient to produce an effective response (i.e., a biological or medical response of a tissue, system, animal or human sought by a researcher, veterinarian, medical doctor or other clinician) upon administration to a patient. The “therapeutically effective amount” will vary depending on, inter alia, the disease and its severity, and on the age, weight, physical condition and responsiveness of the patient to be treated. Furthermore, the “therapeutically effective amount” may vary if nalmefene is combined with one or more other compounds: In such a case the amount of a given compound might be lower, such as a sub-effective amount.

In the present context, “treatment” and “treating” refers to the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relieve the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. In one aspect of the present invention, “treatment” and “treating” refers to prophylactic (preventive) treatment. In another aspect, “treatment and “treating” refers to curative treatment. The patient to be treated is preferably a mammal, in particular a human being.

The term “alcohol dependence” is a commonly known term for a skilled person and is e.g. described in the revised 4^{th} edition of the Diagnostic and Statistical Manual of Mental Disorders (DSM-IV-TR) (Diagnostic and Statistical Manual of Mental Disorders, 4^{th} edition text revision, American Psychiatric Publishing, 2000). As used herein, the term “alcohol dependence” is defined as the presence of three or more of the seven areas of life impairment related to alcohol in the same 12-month period. These impairments include 1) tolerance, 2) withdrawal, 3) the alcohol is often taken in larger amounts or over a longer period than was intended, 4) persistent desire or unsuccessful efforts to cut down or control alcohol intake, 5) a great deal of time is spent in activities necessary to obtain alcohol, intake alcohol, or recover from its effects, 6) important social, occupational, or recreational activities are given up or reduced because of alcohol consumption, 7) alcohol use is continued despite knowledge of having a persistent or recurrent physical or psychological problem that is likely to have been caused or exacerbated by alcohol consumption.

The term “anxiety disorder” is described in DSM-IV-TR and refers to a variety of conditions characterized by a disturbance in mood as the main feature. In the present context, anxiety disorders include Acute Stress Disorder, Agoraphobia, Anxiety, Anxiety Disorder, Emotional Distress, Generalised Anxiety Disorder, Nervousness, Nosophobia, Obsessive-Compulsive Disorder, Panic Attack, Panic Disorder, Panic Disorder With Agoraphobia, Post-Traumatic Stress Disorder, Social Phobia, Stress and Tension.

In the present context, “patients with co-morbid anxiety disorder” refers to patients who are alcohol dependent and at the same time have an anxiety disorder. In one embodiment, said anxiety disorder is caused by said alcohol dependence e.g. said anxiety disorder is an alcohol-induced anxiety disorder. In one embodiment, said alcohol dependence is caused by said anxiety disorder. In one embodiment said alcohol dependence and said anxiety disorder are not causally related to each other.

The term “alcohol induced anxiety disorder” is described in DSM-IV-TR and refers to a disorder characterized by prominent symptoms of anxiety that are judged to be a direct physiological consequence of alcohol abuse.

The term “selective serotonin reuptake inhibitor” (SSRI) means an inhibitor of the monoamine transporters which has stronger affinity effect at the serotonin transporter than the dopamine and the noradrenaline transporters.

The term “serotonin and noradrenaline reuptake inhibitor” (SNRI) means an inhibitor of the monoamine transporters which has an effect both at the serotonin transporter and at the noradrenaline transporter.

The term “POMS” is an abbreviation of “profile of mood states” and refers to a self-report inventory scale developed to assess the effect of e.g. new medication on mood states and mood changes. The scale measures six domains: Tension-Anxiety, Depression-Rejection, Anger-Hostility, Vigour-Activity, Fatigue-Inertia, and Confusion-Bewilderment. A total mood disturbance (TMD) score can be calculated. In general, a lower POMS score indicates a better mood state than a higher score except for vigour-activity, for which a higher POMS score indicates a better mood state. The scale has been described e.g. by McNair et al., Profile of mood states. San Diego, Calif.: Educational and Industrial Testing Service and by Nyenhios and Yamamoto, J. Clin. Psychology, (1999), Vol. 55(1): 79-86.

“MedDRA” is an abbreviation of Medical Dictionary for Regulatory Activities which is a clinically validated international medical terminology dictionary (and thesaurus) used by regulatory authorities in the pharmaceutical industry during the regulatory process, from premarketing to post-marketing activities, and for data entry, retrieval, evaluation, and presentation. In addition, it is the adverse event classification dictionary endorsed by the International Conference on Harmonisation of Technical Requirements for Registration of Pharmaceuticals for Human Use (ICH).

### DETAILED DESCRIPTION OF THE INVENTION

The efficacy of nalmefene in the reduction of alcohol consumption in patients with alcohol dependence (DSM-IV) has been evaluated in studies 12014A, 12023A and 12013A. The efficacy of nalmefene was measured using two co-primary endpoints: the change from baseline in the monthly number of heavy drinking days (HDDs) and the change from baseline in the average daily total alcohol consumption (TAC). In the total patient group, nalmefene was superior to placebo in reducing the number of HDDs and in reducing TAC.

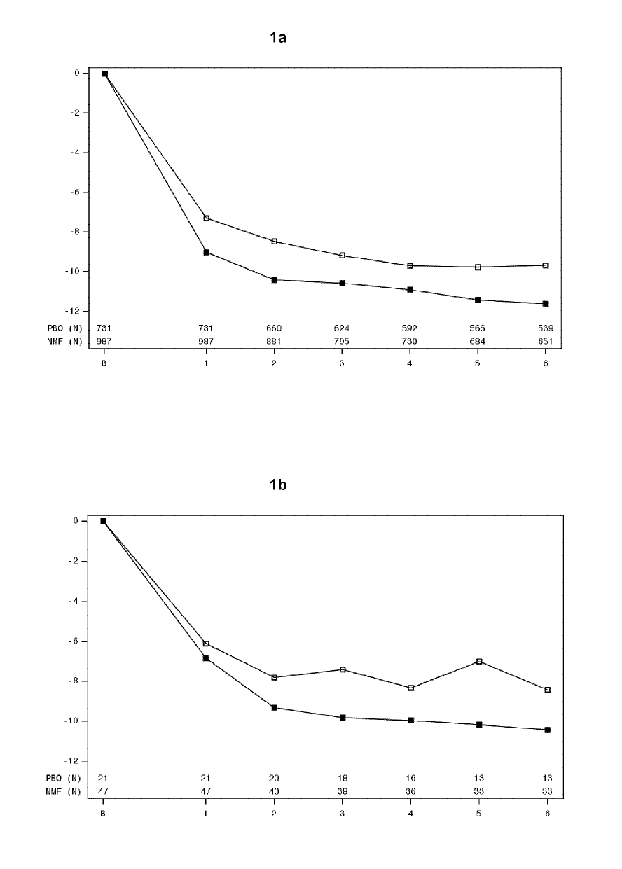

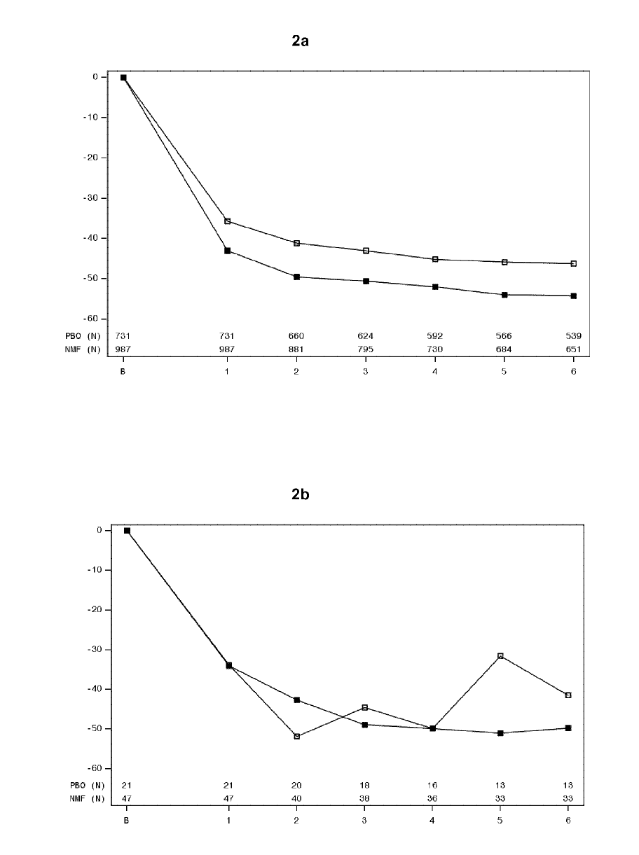

The inventors have found that nalmefene significally reduced the alcohol consumption in patients with an anxiety disorder at baseline. The effect of nalmefene and the effect of placebo on both HDDs and TAC was more or less at the same level as in patients without an anxiety disorder at baseline (FIGS. 1-2).

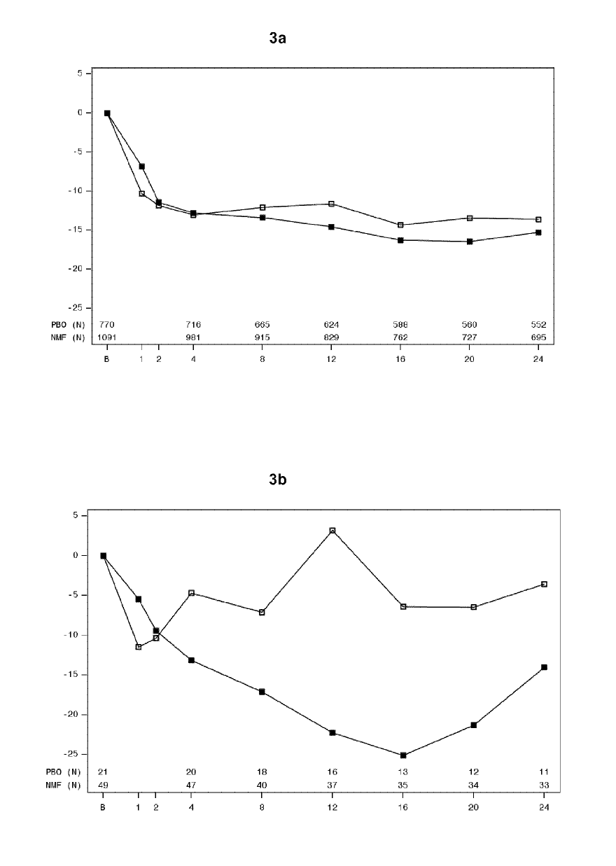

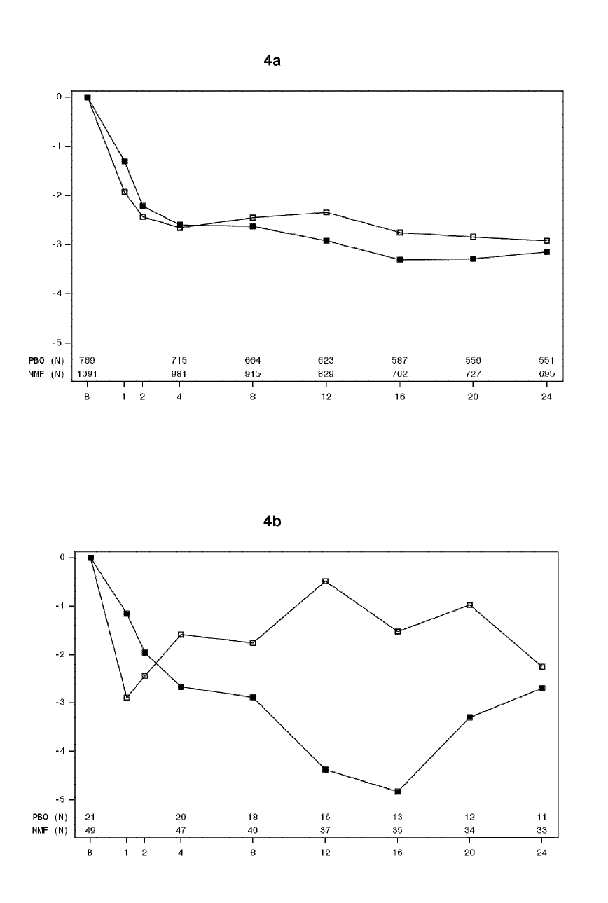

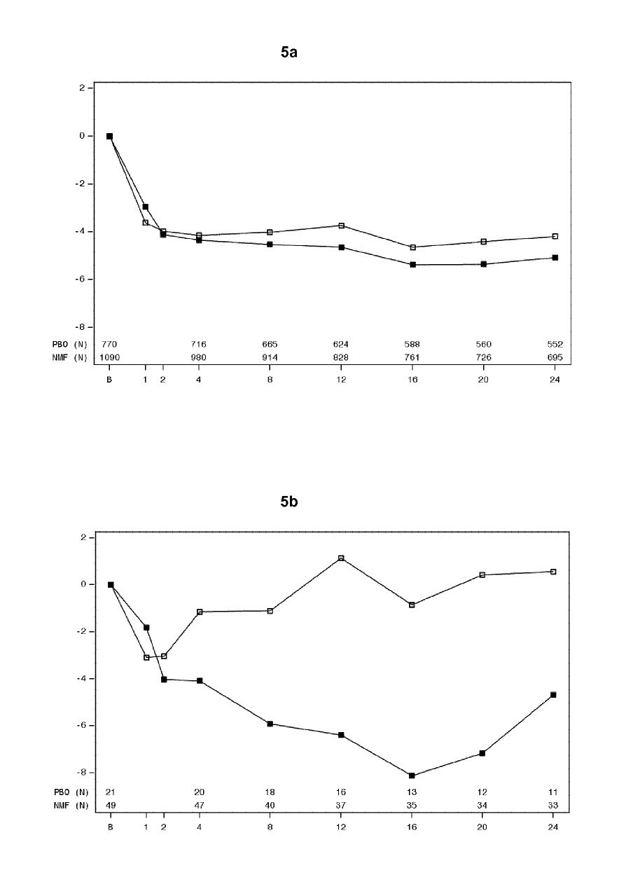

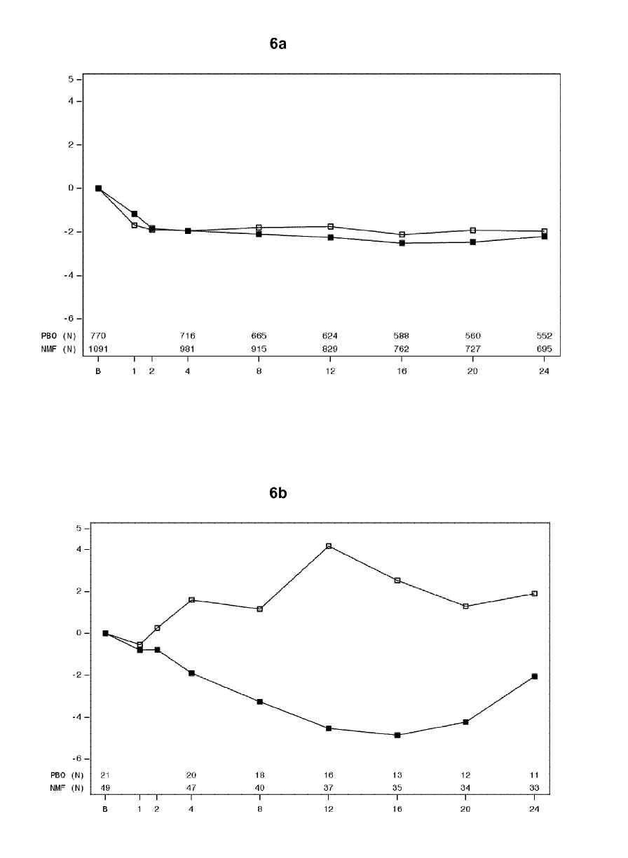

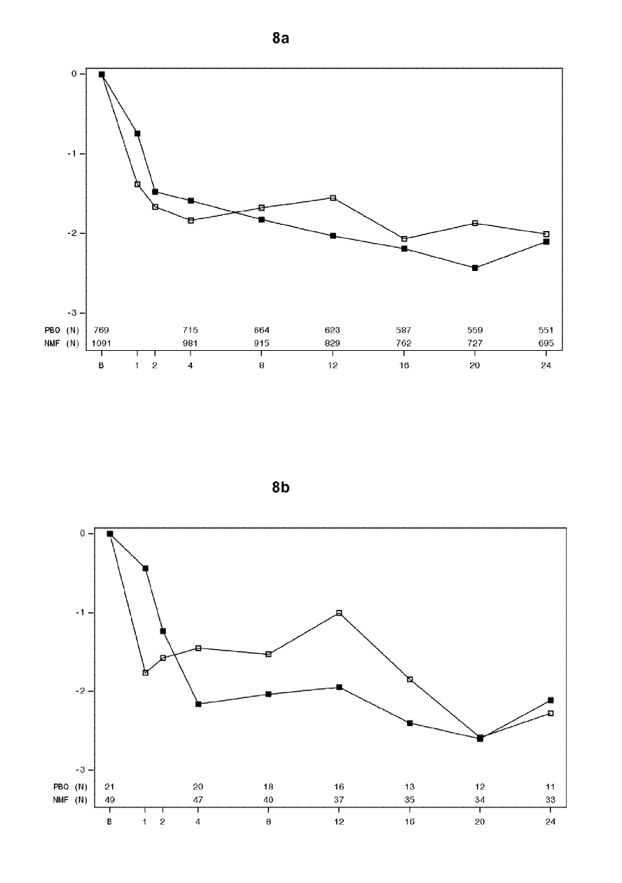

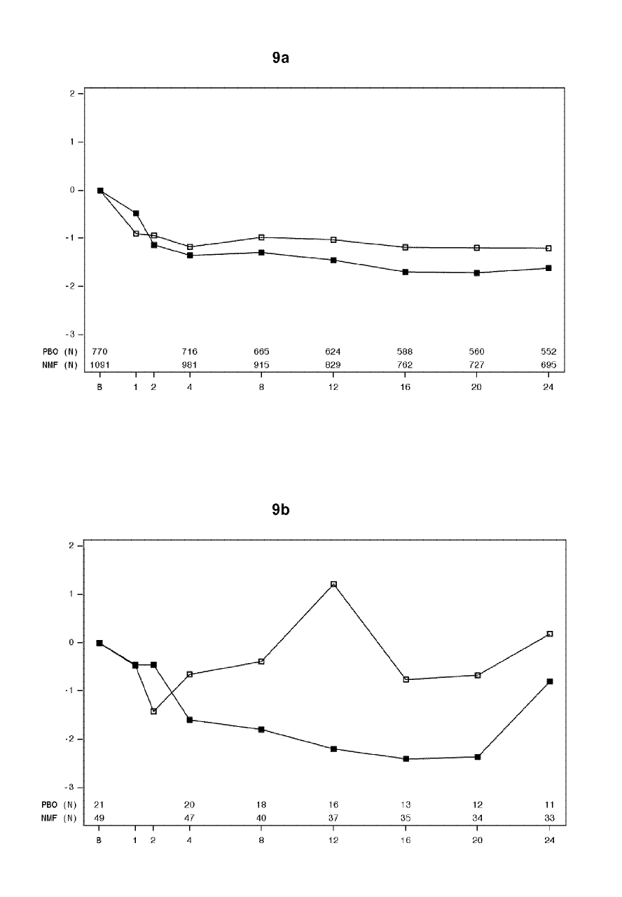

Assessment of POMs scores in Studies 12014A, 12023A and 12013A was used to evaluate the effect of nalmefene on mood states and mood changes throughout the study. The inventors of the present invention surprisingly found that nalmefene has an effect on the POMS scores in patients with an anxiety disorder. Tables 5 and 7 indicate that patients with an anxiety disorder at baseline had higher POMS scores at baseline when compared to those patients without anxiety disorders. The change in POMS scores from baseline are illustrated in FIGS. 3-9. FIGS. 3a-9a indicates that in patients without an anxiety disorder at baseline, the pattern in POMS score was stabile throughout the study with no pronounced difference between nalmefene and placebo. FIGS. 3b-9b indicates that the patients with an anxiety disorder at baseline who received nalmefene had a better POMS score at the end of the study than the patients with an anxiety disorder at baseline who received placebo. In particular FIGS. 3b, 4b, 5b, 6b and 9b representing total mood disturbance, tension-anxiety, depression-rejection, anger-hostility and confusion, respectively, indicates better POMS scores in weeks 4-24 in patients who received nalmefene compared to patients who received placebo. A remarkable improvement is seen over the weeks 8-20. Overall, the POMS data indicates that the general mood state improves in patients with an anxiety disorder when they are treated with nalmefene.

Accordingly, in one embodiment, the present invention therefore relates to nalmefene for treatment of an anxiety disorder. In one embodiment, the invention relates to nalmefene for use in the treatment of patients with alcohol dependence who have a co-morbid anxiety disorder. In one embodiment, the invention relates to nalmefene for reduction of alcohol consumption in patients with alcohol dependence who have a co-morbid anxiety disorder. In one embodiment, the invention relates to nalmefene for treatment of an anxiety disorder in patients with alcohol dependence who have a co-morbid anxiety disorder. In a further embodiment, the invention relates to nalmefene for use in the reduction of alcohol consumption and for treatment of an anxiety disorder in patients with alcohol dependence who have a comorbid anxiety disorder.

In one embodiment, nalmefene is used as the sole active ingredient for the treatment of an anxiety disorder. In one embodiment, nalmefene is used as the sole active ingredient in the treatment of patients with alcohol dependence who have a co-morbid anxiety disorder.

In one embodiment, nalmefene used in combination with a second compound which is an antianxiety agent such as a serotonin reuptake inhibitor, or any other compound which causes an elevation in the level of extracellular serotonin, for the treatment of an anxiety disorder. In another embodiment, nalmefene is used in combination with a second compound which is an antianxiety agent such as a serotonin reuptake inhibitor, or any other compound which causes an elevation in the level of extracellular serotonin, in the treatment of patient of patients with alcohol dependence who have a co-morbid anxiety disorder.

The present invention also relates to a pharmaceutical composition comprising nalmefene and a second compound, which is an antianxiety agent such as a serotonin reuptake inhibitor, or any other compound which causes an elevation in the level of extracellular serotonin, and optionally acceptable carriers or diluents.

Further assessment of the effect of nalmefene on the treatment of anxiety disorders can be performed by testing nalmefene in non-clinical models e.g. such the marble burying test as outlined in Example 4. In such models nalmefene can be tested as the sole active substance as well as in combination with other compounds.

According to the present invention, nalmefene or a pharmaceutically acceptable salt thereof may be administered in any suitable way, e.g. orally, transmucosally or parenterally, and it may be presented in any suitable form for such administration, e.g. in the form of tablets, capsules, powders, syrups or solutions or dispersions for injection. In another embodiment, and in accordance with the purpose of the present invention, nalmefene is administered in the form of a solid pharmaceutical entity, suitably as a tablet or a capsule or in the form of a suspension, solution or dispersion for injection. Additionally, nalmefene may be administered with a pharmaceutically acceptable carrier, such as an adjuvant and/or diluent.

Methods for the preparation of solid or liquid pharmaceutical preparations are well known in the art. See e.g. Remington: The Science and Practice of Pharmacy, 21^{st} ed., Lippincott Williams & Wilkins (2005). Tablets may thus be prepared by mixing the active ingredients with an ordinary carrier, such as an adjuvant and/or diluent, and subsequently compressing the mixture in a tableting machine. Non-limiting examples of adjuvants and/or diluents include: corn starch, lactose, talcum, magnesium stearate, gelatin, lactose, gums, and the like. Any other adjuvant or additive such as colorings, aroma, and preservatives may also be used provided that they are compatible with the active ingredients. The pharmaceutical compositions of the invention thus typically comprise an effective amount of nalmefene and one or more pharmaceutically acceptable carrier. A suitable oral formulation of nalmefene is described in WO 2012/059103.

Without limiting the invention in any way, it is intended that any one of the aspects or embodiments of this patent application is suitable for the medicaments or pharmaceutical compositions described herein.

Nalmefene may be administered as an oral dose form, such as a solid oral dose form, typically tablets or capsules, or as a liquid oral dose form. Nalmefene may be administered in an immediate release dosage form or a controlled or sustained release dosage form. Nalmefene may be conveniently administered orally in unit dosage forms, such as tablets or capsules, containing the active ingredient in an amount from about 1 to about 100 mg, such as from 5 to 50 mg. Typically, the pharmaceutical composition comprises from 10 mg to 20 mg, such as about 10 mg, about 11 mg, about 12 mg, about 13 mg, about 14 mg, about 15 mg, about 16 mg, about 17 mg, about 18 mg, about 19 mg or about 20 mg of nalmefene. In a preferred embodiment, the pharmaceutical composition comprises about 18 mg of nalmefene. In one embodiment, the unit dosage form comprises nalmefene in a therapeutically effective amount.

In one embodiment, nalmefene is taken as-needed, that is, on each day a patient perceives a risk of drinking alcohol, one dose of nalmefene should be taken, preferably 1-2 hours prior to anticipated time of drinking. In one embodiment, if the patient has started drinking alcohol without taking nalmefene, the patient should take one dose of nalmefene as soon as possible after that.

Nalmefene according to the present invention is intended to be used for dosing in humans who are adults or adolescents.

In one embodiment, nalmefene is in the form of the hydrochloride dihydrate.

According to the invention, nalmefene can be used in combination with a second compound which is an antianxiety agent such as a serotonin reuptake inhibitor, or any other compound which causes an elevation in the level of extracellular serotonin. Said antianxiety agent may e.g. be selected from the following compounds; citalopram, escitalopram, fluoxetine, sertraline, paroxetine, fluvoxamine, venlafaxine, duloxetine, dapoxetine, nefazodone, imipramine, femoxetine, clomipramine, agomelatine and buspirone. The compounds mentioned above may be used in the form of the base or a pharmaceutically acceptable salt, such as an acid addition salt, thereof. The above list of antianxiety agents may not be construed as limiting.

Antianxiety agents including the SSRIs, SNRIs and other agents specifically mentioned hereinabove, differ both in molecular weight and in activity. As a consequence, the amount of said second compound used in combination therapy depends on the nature of said second compound. In one embodiment of the invention, said second compound is administered at lower doses than required when the compound is used alone. In another embodiment, said second compound is administered in normal therapeutic doses.

To prepare the pharmaceutical compositions of this invention, an appropriate amount of the active ingredient(s), in salt form or base form, is combined in an intimate admixture with a pharmaceutically acceptable carrier, which can take a wide variety of forms depending on the form of preparation desired for administration. These pharmaceutical compositions are desirably in unitary dosage form suitable for administration orally, rectally, percutaneously or by parenteral injection. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed.

It is especially advantageous to formulate the aforementioned pharmaceutical compositions in dosage unit form for ease of administration and uniformity of dosage. As used in the specification and claims, unit dosage form refers to physically discrete units suitable as unitary dosages, each unit containing a predetermined quantity of active ingredient(s) calculated to produce the desired therapeutic effect, in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

Nalmefene may be administered before, during or after the administration of said second compound provided that the time between the administration of nalmefene and the administration of said second compound is such that ingredients are allowed to act synergistically on the CNS. When simultaneous administration of nalmefene and said second compound is envisaged, a composition containing both said second compound and nalmefene may be particularly convenient. Alternatively, nalmefene and said second compound may be administered separately in the form of suitable compositions. The compositions may be prepared as described hereinabove.

The present invention also comprises products containing nalmefene and a second compound which is an antianxiety agent as a combination preparation for simultaneous, separate or sequential use in psychiatric drug therapy. Such products may comprise, for example, a kit comprising discrete unit dosage forms containing nalmefene and discrete unit dosage forms containing an antianxiety agent, all contained in the same container or pack, e.g. a blister pack.

The second compound contained in the above mentioned preparations for simultaneous or sequential administration is an antianxiety agent which may e.g. be selected from a serotonin reuptake inhibitor such as an SSRI or another compound causing an elevation in the level of extracellular serotonin.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the various aspects, embodiments, implementations and features of the invention mentioned herein may be claimed separately, or in any combination.

### Embodiments According to the Invention

In the following, embodiments of the invention are disclosed. The first embodiment is denoted E1, the second embodiment is denoted E2 and so forth.
E1. Nalmefene for use in the treatment of an anxiety disorder.
E2. Nalmefene for use in the treatment of a patient with alcohol dependence who has a co-morbid anxiety disorder.
E3. Nalmefene according to embodiment 1 or 2 for use in the treatment of an anxiety disorder in a patient with alcohol dependence who has a co-morbid anxiety disorder.
E4. Nalmefene for use in the reduction of alcohol consumption and for use in the treatment of an anxiety disorder according to embodiment 3 in a patient with alcohol dependence who has a co-morbid anxiety disorder.
E5. Nalmefene according to any of embodiments 1-4, wherein said anxiety disorder or comorbid anxiety disorder is an alcohol induced anxiety disorder.
E6. Nalmefene according to any of embodiments 2-4, wherein said alcohol dependence is caused by said anxiety disorder.
E7. Nalmefene according to any of embodiments 2-4, wherein said alcohol dependence and said anxiety disorder are not causally related to each other.
E8. Nalmefene according to any of embodiments 1-7, wherein said anxiety disorder or comorbid anxiety disorder is selected from Acute Stress Disorder, Agoraphobia, Anxiety, Anxiety Disorder, Emotional Distress, Generalised Anxiety Disorder, Nervousness, Nosophobia, Obsessive-Compulsive Disorder, Panic Attack, Panic Disorder, Panic Disorder With Agoraphobia, Post-Traumatic Stress Disorder, Social Phobia, Stress, and Tension.
E9. Nalmefene according to any of embodiments 1-8, wherein said nalmefene is the sole active ingredient used in the treatment of said anxiety disorder and/or in the reduction of said alcohol consumption.
E10. Nalmefene according to any of embodiments 1-9, wherein said patient is further treated with a second compound which is an antianxiety agent.
E11. Nalmefene according to embodiment 10, wherein said second compound is selected from a serotonin reuptake inhibitor, or any other compound which causes an elevation in the level of extracellular serotonin.
E12. Nalmefene according to embodiment 11, wherein said serotonin reuptake inhibitor is a selective serotonin reuptake inhibitor.
E13. Nalmefene according to embodiment 11, wherein said second compound is selected from citalopram, escitalopram, fluoxetine, sertraline, paroxetine, fluvoxamine, venlafaxine, duloxetine, dapoxetine, nefazodone, imipramine, femoxetine, clomipramine, agomelatine, and buspirone or a pharmaceutically acceptable salt of any of these compounds.
E14. Nalmefene according to any of embodiments 10-13, wherein said nalmefene and said second compound are contained in the same unit dosage form.
E15. Nalmefene according to any of embodiments 10-13, wherein said nalmefene and said second compound are contained in the separate unit dosage forms.
E16. Nalmefene according to any of embodiments 2-15, wherein said patient has at least a medium drinking risk level.
E17. Nalmefene according to embodiment 16, wherein said patient has a high drinking risk level.
E18. Nalmefene according to embodiment 17, wherein said patient has a drinking risk level corresponding to consumption >60 g/day of pure alcohol for men and >40 g/day for women.
E19. Nalmefene according to any of embodiments 1-18, wherein said nalmefene is to be used as-needed.
E20. Nalmefene according to any of embodiments 1-19, wherein said nalmefene is used in a dose of 10-20 mg such as 10 mg, 11 mg, 12 mg, 13 mg, 14 mg, 15 mg, 16 mg, 17 mg, 18 mg, 19 mg or 20 mg.
E21. Nalmefene according to embodiment 20, wherein said nalmefene is used in a dose of 18 mg.
E22. Nalmefene according to any of embodiments 1-21, wherein said nalmefene is used in the form of a pharmaceutically acceptable acid addition salt.
E23. Nalmefene according to embodiment 22, wherein said nalmefene is used in the form of a hydrochloride salt.
E24. Nalmefene according to embodiment 23, wherein said nalmefene is used in the form of the hydrochloride dihydrate.
E25. Nalmefene according to embodiment 24, wherein said nalmefene is used in a crystalline form.
E26. Nalmefene according to any of embodiments 1-25, wherein said nalmefene is contained in an oral dose form such as tablets or capsules.
E27. A pharmaceutical composition comprising nalmefene and a second compound which is an antianxiety agent, and optionally acceptable carriers or diluents.
E28. The pharmaceutical composition according to embodiment 27, wherein said second compound is a serotonin reuptake inhibitor, or any other compound which causes an elevation in the level of extracellular serotonin.
E29. A kit comprising nalmefene together with a second compound, which is an antianxiety agent.
E30. The kit according to embodiment 29, wherein said second compound is a serotonin reuptake inhibitor, or any other compound which causes an elevation in the level of extracellular serotonin.
E31. The pharmaceutical composition according embodiment 28 or the kit according to embodiment 30, wherein said serotonin reuptake inhibitor is a selective serotonin reuptake inhibitor.
E32. The pharmaceutical composition according embodiment 27 or the kit according to embodiment 29, wherein said second compound is selected from citalopram, escitalopram, fluoxetine, sertraline, paroxetine, fluvoxamine, venlafaxine, duloxetine, dapoxetine, nefazodone, imipramine, femoxetine, clomipramine, agomelatine, buspirone or a pharmaceutically acceptable salt of any of these compounds.
E33. The kit according to any of embodiments 29-32, which is adapted for sequential administration of said nalmefene and said second compound.
E34. The pharmaceutical composition or the kit according to any of embodiments 27-32, which is adapted for simultaneous administration of said nalmefene and said second compound.
E35. The pharmaceutical composition or the kit according to embodiment 34, wherein said nalmefene and said second compound are contained in the same unit dosage form.
E36. The pharmaceutical composition or the kit according to any of embodiments 27-35, wherein said nalmefene is present in a dose of 10-20 mg such as 10 mg, 11 mg, 12 mg, 13 mg, 14 mg, 15 mg, 16 mg, 17 mg, 18 mg, 19 mg or 20 mg.
E37. The pharmaceutical composition or the kit according to embodiment 36, wherein said nalmefene is present in a dose of 18 mg.
E38. The pharmaceutical composition or the kit according to any of embodiments 27-37, wherein said nalmefene is present in the form of a pharmaceutically acceptable acid addition salt.
E39. The pharmaceutical composition or the kit according to embodiment 38, wherein said nalmefene is present in the form of a hydrochloride salt.
E40. The pharmaceutical composition or the kit according to embodiment 39, wherein said nalmefene is present in the form of the hydrochloride dihydrate.
E41. The pharmaceutical composition or the kit according to embodiment 40, wherein said nalmefene is present in a crystalline form.
E42. A method for the treatment of an anxiety disorder, which method comprises administering a pharmaceutically acceptable amount of nalmefene to a patient in need thereof.
E43. A method for reduction of alcohol consumption and for the treatment of an anxiety disorder, which method comprises administering a pharmaceutically acceptable amount of nalmefene to a patient in need thereof.
E44. The method according to any of embodiments 42-43, wherein said patient is alcohol dependent and has a co-morbid anxiety disorder.
E45. The method according to embodiment 44, wherein said anxiety disorder or co-morbid anxiety disorder is an alcohol induced anxiety disorder.
E46. The method according to embodiment 44, wherein said alcohol dependence is caused by said anxiety disorder.
E47. The method according to embodiment 44, wherein said alcohol dependence and said anxiety disorder are not causally related to each other.
E48. The method according to any of embodiments 42-47, wherein said anxiety disorder or co-morbid anxiety disorder is selected from Acute Stress Disorder, Agoraphobia, Anxiety, Anxiety Disorder, Emotional Distress, Generalised Anxiety Disorder, Nervousness, Nosophobia, Obsessive-Compulsive Disorder, Panic Attack, Panic Disorder, Panic Disorder With Agoraphobia, Post-Traumatic Stress Disorder, Social Phobia, Stress, and Tension.
E49. The method according to any of embodiments 42-48, wherein said nalmefene is the sole active ingredient used in the treatment of said anxiety disorder and/or in the reduction of said alcohol consumption.
E50. The method according to any of embodiments 42-49, which method further comprises administering a pharmaceutically acceptable amount of a second compound which is an antianxiety agent.
E51. The method according to embodiment 50, wherein said second compound is a serotonin reuptake inhibitor, or any other compound which causes an elevation in the level of extracellular serotonin.
E52. The method according to embodiment 51, wherein said serotonin reuptake inhibitor is a selective serotonin reuptake inhibitor.
E53. The method according embodiment 50, wherein said second compound is selected from citalopram, escitalopram, fluoxetine, sertraline, paroxetine, fluvoxamine, venlafaxine, duloxetine, dapoxetine, nefazodone, imipramine, femoxetine, clomipramine, agomelatine, buspirone or a pharmaceutically acceptable salt of any of these compounds.
E54. The method according to any of embodiments 50-53, wherein said nalmefene and said second compound are contained in the same unit dosage form.
E55. The method according to any of embodiments 50-53, wherein said nalmefene and said second compound are contained in separate unit dosage forms.
E56. The method according to any of embodiments 42-55, wherein said patient has at least a medium drinking risk level.
E57. The method according to embodiment 56, wherein said patient has a high drinking risk level.
E58. The method according to embodiment 57, wherein said patient has a drinking risk level corresponding to consumption >60 g/day of pure alcohol for men and >40 g/day for women.
E59. The method according to any of embodiments 42-58, wherein said nalmefene is administered as-needed.
E60. The method according to any of embodiments 42-59, wherein said nalmefene is administered in a dose of 10-20 mg such as 10 mg, 11 mg, 12 mg, 13 mg, 14 mg, 15 mg, 16 mg, 17 mg, 18 mg, 19 mg or 20 mg.
E61. The method according to embodiment 60, wherein said nalmefene is administered in a dose of 18 mg.
E62. The method according to any of embodiments 42-61, wherein said nalmefene is administered in the form of a pharmaceutically acceptable acid addition salt.
E63. The method according to embodiment 62, wherein said nalmefene is administered in the form of a hydrochloride salt.
E64. The method according to embodiment 63, wherein said nalmefene is administered in the form of the hydrochloride dihydrate.
E65. The method according to embodiment 64, wherein said nalmefene is administered in a crystalline form.
E66. The method according to any of embodiments 42-65, wherein said nalmefene is contained in an oral dose form such as tablets or capsules.

### EXAMPLES

The invention will be illustrated by the following non-limiting examples.

### Clinical Assessment

The diagnosis of alcohol dependence was based on the DSM-IV-TR criteria. For this purpose, the investigator interviewed the patient in a structured way by using the Mini International Neuropsychiatric Interview (MINI) standardized interview (Lecrubier Y. et al. The Mini International Neuropsychiatric Interview (MINI). A short diagnostic structured interview: Reliability and validity according to the CIDI. European Psychiat. (1997), 12:224-31). The M.I.N.I. is designed as a brief structured interview for the major Axis I psychiatric disorders in DSM-IV. Its use permits a standardized assessment of the diagnostic criteria. The M.I.N.I. interview was used at the screening visit. Clinicians used it after a training session. The M.I.N.I. approach can also be used to select patients with an anxiety disorder at baseline.

Another approach to identify patients with an anxiety disorder is by defining Anxiety Disorder at baseline as any ongoing medical history coded by the Medical Dictionary for Regulatory Activities (MedDRA) Preferred Term as ‘Acute Stress Disorder’, ‘Agoraphobia’, ‘Anxiety’, ‘Anxiety Disorder’, ‘Emotional Distress’, ‘Generalised Anxiety Disorder’, ‘Nervousness’, ‘Nosophobia’, ‘Obsessive-Compulsive Disorder’, ‘Panic Attack’, ‘Panic Disorder’, ‘Panic Disorder With Agoraphobia’, ‘Post-Traumatic Stress Disorder’, ‘Social Phobia’ and/or ‘Stress and Tension’. In examples 1 and 2 below patients classified with an anxiety disorder at baseline were selected based on said MedDRA terms.

### Example 1

### Clinical Efficacy on the Reduction of Alcohol Consumption

The efficacy of nalmefene on the reduction of alcohol consumption in patients with alcohol dependence (DSM-IV) was evaluated in two efficacy studies (Study 12014A and Study 12023A) and a safety study (Study 12013A). All studies were multi-national, multi-site, randomised, double blind, two parallel group, placebo controlled studies. The efficacy was evaluated over 24 weeks of treatment. The studies included outpatients, aged ≧18 years, with a primary diagnosis of alcohol dependence. A patient was eligible for participation in the study if, in the 4 weeks preceding the Screening Visit, he/she had: ≧6 HDDs, ≦14 consecutive abstinent days, did not have serum aspartate aminotransferase (ASAT) and/or serum alanine aminotransferase (ALAT) values >3 times upper limit of the reference range, that are in the investigator's opinion clinically significant. Patients with psychiatric co-morbidity (that is, patients who used stable doses of antipsychotics and/or certain antidepressants) were also included unless the treatment of the psychiatric comorbidity had to take priority over treatment of the drinking problem, or was likely to interfere with study treatment or impairs treatment compliance.

The studies included in total 1997 patients, 1173 of whom were treated with nalmefene 18 mg in an as-needed dosing regimen. A motivational and adherence enhancing intervention was administered to all the patients to support the patients in changing their behavior and to enhance adherence to treatment.

The efficacy of nalmefene on the reduction of alcohol consumption was measured using two co-primary endpoints: the change in the monthly number of heavy drinking days (HDDs) and the change in the average daily total alcohol consumption (TAC). A HDD was defined as a day with a consumption ≧60 g alcohol for men and ≧40 g for women. The change in HDD and TAC over time in patients treated with nalmefene or placebo is reflected in FIGS. 1-2 indicating that the difference between nalmefene and placebo measured in HDDs and TAC at week 24 was merely at the same level in the group of patients with a anxiety disorder at baseline as in patients without a anxiety disorder at baseline.

### Example 2

### Clinical Efficacy Measured by POMS Score

Assessment of POMs scores in Studies 12014A, 12023A and 12013A was used to evaluate the effect of nalmefene on mood states and mood changes throughout the study. Tables 5 and 7 indicate that patients with an anxiety disorder at baseline had higher POMS scores at baseline when compared to those without an anxiety disorder. The change in POMS scores from baseline are illustrated in FIGS. 3-9. FIGS. 3a-9a indicates that in patients without an anxiety disorder at baseline, the pattern in POMS score was stabile throughout the study with no pronounced difference between nalmefene and placebo. FIGS. 3b-9b indicates that the patients with an anxiety disorder at baseline who received nalmefene had a better POMS score at the end of the study than the patients with an anxiety disorder at baseline who received placebo.

In particular FIGS. 3b, 4b, 5b, 6b and 9b representing total mood disturbance, tension-anxiety, depression-rejection, anger-hostility and confusion, respectively, indicates better POMS scores in weeks 4-24 in patients who received nalmefene compared to patients who received placebo.

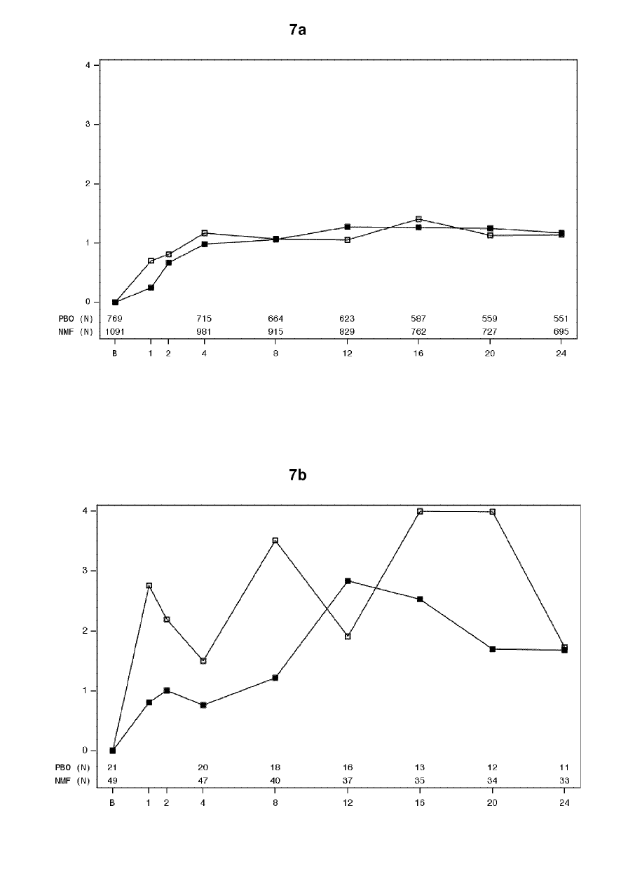

In general a lower POMS score indicates a better mood state than a higher score except for vigour, illustrated in FIGS. 7a and 7b, wherein a higher POMS score indicates a better mood state.

The demographic data and baseline characteristics for the studies 12014A, 12023A and 12013A are provided in tables 4-7 below wherein the medical history according to MedDRA was used for patient selection.

TABLE 4
Patient Demographics (APRS) -
Patients without an anxiety disorder at baseline.
Studies 12014A, 12023A and 12013A pooled.

| | | Placebo | Nalmefene | Total |
|---|---|---|---|---|
| Number of Patients | | 802 | 1123 | 1925 |
| Age | N | 802 | 1123 | 1925 |
| | Mean | 47.06 | 46.20 | 46.56 |
| | SD | 11.11 | 11.18 | 11.16 |
| | Min | 18.00 | 19.00 | 18.00 |
| | Max | 75.00 | 77.00 | 77.00 |
| | Median | | | |
| Age Group n | <25 | 16 | 24 | 40 |
| | 25-34 | 105 | 158 | 263 |
| | 35-44 | 195 | 317 | 512 |
| | 45-54 | 273 | 360 | 633 |
| | 55-64 | 172 | 196 | 368 |
| | >=65 | 41 | 68 | 109 |
| Sex n | F | 231 | 287 | 518 |
| | M | 571 | 836 | 1407 |
| Race n | Asian | 0 | 3 | 3 |
| | Black | 3 | 4 | 7 |
| | Caucasian | 797 | 1115 | 1912 |
| Race n | Other | 2 | 1 | 3 |

TABLE 5
Baseline Characteristics (APRS) -
Patients without an anxiety disorder at baseline.
Studies 12014A, 12023A and 12013A pooled.

| | | Placebo | Nalmefene | Total |
|---|---|---|---|---|
| Monthly number of HDDs | N | 802 | 1121 | 1923 |
| | Mean | 17.77 | 17.18 | 17.42 |
| | SD | 7.13 | 7.30 | 7.23 |
| | Min | 0.00 | 0.00 | 0.00 |
| | Max | 28.00 | 28.00 | 28.00 |
| | Median | 17.00 | 16.00 | 16.00 |
| Total Alcohol Consumption | N | 802 | 1121 | 1923 |
| | Mean | 82.33 | 79.96 | 80.95 |
| | SD | 44.37 | 43.74 | 44.01 |
| | Min | 17.00 | 7.00 | 7.00 |
| | Max | 380.00 | 447.00 | 447.00 |
| | Median | 72.00 | 69.00 | 70.00 |
| POMS - TMD | N | 800 | 1122 | 1922 |
| | Mean | 36.23 | 36.10 | 36.16 |
| | SD | 36.77 | 36.00 | 36.31 |
| | Min | −28.00 | −25.00 | −28.00 |
| | Max | 206.00 | 169.00 | 206.00 |
| | Median | 28.00 | 27.00 | 27.79 |
| POMS - Tension-Anxiety | N | 799 | 1122 | 1921 |
| | Mean | 11.34 | 11.36 | 11.36 |
| | SD | 6.83 | 6.79 | 6.80 |
| | Min | 0.00 | 0.00 | 0.00 |
| | Max | 35.00 | 35.00 | 35.00 |
| | Median | 10.00 | 10.00 | 10.00 |
| POMS - Depression Rejection | N | 800 | 1121 | 1921 |
| | Mean | 14.07 | 14.04 | 14.05 |
| | SD | 12.23 | 12.06 | 12.13 |
| | Min | 0.00 | 0.00 | 0.00 |
| | Max | 60.00 | 58.00 | 60.00 |
| | Median | 11.00 | 11.00 | 11.00 |
| POMS - Anger-Hostility | N | 800 | 1122 | 1922 |
| | Mean | 9.77 | 9.94 | 9.87 |
| | SD | 7.90 | 7.96 | 7.94 |
| | Min | 0.00 | 0.00 | 0.00 |
| | Max | 41.00 | 47.00 | 47.00 |
| | Median | 8.00 | 8.00 | 8.00 |
| POMS - Vigour | N | 799 | 1122 | 1921 |
| | Mean | 14.29 | 14.61 | 14.48 |
| | SD | 5.91 | 5.77 | 5.83 |
| | Min | 0.00 | 0.00 | 0.00 |
| | Max | 30.00 | 30.00 | 30.00 |
| | Median | 15.00 | 15.00 | 15.00 |
| POMS - Fatigue | N | 799 | 1122 | 1921 |
| | Mean | 7.87 | 7.85 | 7.86 |
| | SD | 6.06 | 5.89 | 5.96 |
| | Min | 0.00 | 0.00 | 0.00 |
| | Max | 28.00 | 27.00 | 28.00 |
| | Median | 7.00 | 7.00 | 7.00 |
| POMS - Confusion | N | 800 | 1122 | 1922 |
| | Mean | 7.35 | 7.52 | 7.45 |
| | SD | 4.70 | 4.52 | 4.59 |
| | Min | 0.00 | 0.00 | 0.00 |
| | Max | 26.00 | 26.00 | 26.00 |
| | Median | 7.00 | 7.00 | 7.00 |

TABLE 6
Patient Demographics (APRS) -
Patients with an anxiety disorder at baseline. Studies
12014A, 12023A and 12013A pooled.

| | | Placebo | Nalmefene | Total |
|---|---|---|---|---|
| Number of Patients | | 22 | 50 | 72 |
| Age | N | 22 | 50 | 72 |
| | Mean | 51.00 | 48.16 | 49.03 |
| | SD | 7.80 | 10.23 | 9.59 |
| | Min | 37.00 | 25.00 | 25.00 |
| | Max | 66.00 | 72.00 | 72.00 |
| | Median | 51.50 | 47.00 | 49.00 |
| Age Group n | 25-34 | 0 | 4 | 4 |
| | 35-44 | 5 | 14 | 19 |
| | 45-54 | 9 | 17 | 26 |
| | 55-64 | 7 | 12 | 19 |
| | >=65 | 1 | 3 | 4 |
| Sex n | F | 8 | 23 | 31 |
| | M | 14 | 27 | 41 |
| Race n | Caucasian | 22 | 50 | 72 |

TABLE 7
Baseline Characteristics (APRS) -
Patients with an anxiety disorder at baseline.
Studies 12014A, 12023A and 12013A pooled.

| | | Placebo | Nalmefene | Total |
|---|---|---|---|---|
| Monthly number of HDDs | N | 22 | 50 | 72 |
| | Mean | 20.55 | 18.06 | 18.82 |
| | SD | 7.10 | 6.71 | 6.88 |
| | Min | 6.00 | 8.00 | 6.00 |
| | Max | 28.00 | 28.00 | 28.00 |
| | Median | 23.00 | 18.00 | 18.00 |
| Total Alcohol Consumption | N | 22 | 50 | 72 |
| | Mean | 103.36 | 82.32 | 88.75 |
| | SD | 61.33 | 48.66 | 53.31 |
| | Min | 39.00 | 22.00 | 22.00 |
| | Max | 300.00 | 209.00 | 300.00 |
| | Median | 84.00 | 67.00 | 76.00 |
| POMS - TMD | N | 22 | 50 | 72 |
| | Mean | 53.49 | 52.32 | 52.67 |
| | SD | 43.37 | 40.66 | 41.31 |
| | Min | −9.00 | −2.00 | −9.00 |
| | Max | 158.88 | 120.00 | 158.00 |
| | Median | 42.00 | 44.00 | 44.00 |
| POMS - Tension-Anxiety | N | 22 | 50 | 72 |
| | Mean | 15.71 | 13.95 | 14.48 |
| | SD | 8.85 | 6.66 | 7.38 |
| | Min | 3.00 | 3.00 | 3.00 |
| | Max | 34.88 | 27.00 | 34.88 |
| | Median | 14.00 | 13.00 | 13.50 |
| POMS - Depression Rejection | N | 22 | 50 | 72 |
| | Mean | 18.54 | 19.40 | 19.14 |
| | SD | 15.24 | 12.80 | 13.49 |
| | Min | 1.00 | 2.00 | 1.00 |
| | Max | 56.00 | 47.14 | 56.00 |
| | Median | 16.50 | 14.40 | 15.50 |
| POMS - Anger-Hostility | N | 22 | 50 | 72 |
| | Mean | 10.73 | 13.23 | 12.46 |
| | SD | 9.22 | 10.02 | 9.79 |
| | Min | 0.00 | 0.00 | 0.00 |
| | Max | 35.00 | 40.00 | 40.00 |
| | Median | 8.50 | 11.00 | 10.50 |
| POMS - Vigour | N | 22 | 50 | 72 |
| | Mean | 10.68 | 12.70 | 12.08 |
| | SD | 6.31 | 6.52 | 6.48 |
| | Min | 1.00 | 2.00 | 1.00 |
| | Max | 25.00 | 24.00 | 25.00 |
| | Median | 9.50 | 11.50 | 10.50 |
| POMS - Fatigue | N | 22 | 50 | 72 |
| | Mean | 9.82 | 9.40 | 9.53 |
| | SD | 6.28 | 7.03 | 6.77 |
| | Min | 1.00 | 0.00 | 0.00 |
| | Max | 25.00 | 25.00 | 25.00 |
| | Median | 9.00 | 8.00 | 9.00 |
| POMS - Confusion | N | 22 | 50 | 72 |
| | Mean | 9.38 | 9.04 | 9.14 |
| | SD | 4.74 | 5.33 | 5.12 |
| | Min | 1.00 | 1.00 | 1.00 |
| | Max | 18.00 | 18.00 | 18.00 |
| | Median | 8.00 | 8.50 | 8.00 |

### Example 3

### Clinical Efficacy Measured by SF-36 Mental Component Summary (FAS, OC)

Another method for measuring a patient's health status is by the SF-36 which is a patient-reported outcome developed as a general measure of perceived health status. The mental component summary score focuses on mental aspects of health related quality of life. Higher scores correspond to better health status or well-being.

Data on the mental component summary score are presented in Table 8 below. The difference between nalmefene and placebo in the change from baseline to Week 12 and Week 24 was more pronounced in patients with an anxiety disorder at baseline than in the patients without an anxiety disorder at baseline.

TABLE 8
Change from Baseline to Week 12 and Week 24 in SF-36 Mental
Component Summary (FAS, OC) by anxiety disorder at baseline -
Studies 12014A, 12023A and 12013A pooled.

| Anxiety disorder | | | | | | |
|---|---|---|---|---|---|---|
| at baseline | Baseline | | Change to Week 12 | | Change to Week 24 | |
| Treatment Group | N | Mean ± SE | N | Mean ± SE | N | Mean ± SE |
| No | | | | | | |
| Placebo | 715 | 40.6 ± 0.46 | 615 | 3.58 ± 0.44 | 522 | 4.59 ± 0.47 |
| Nalmefene | 977 | 40.6 ± 0.38 | 828 | 4.77 ± 0.37 | 628 | 5.65 ± 0.43 |
| Yes | | | | | | |
| Placebo | 21 | 35.9 ± 2.42 | 18 | 0.14 ± 2.81 | 25 | 0.75 ± 3.87 |
| Nalmefene | 45 | 34.1 ± 1.82 | 41 | 6.23 ± 1.47 | 39 | 7.56 ± 2.24 |

### Non-Clinical Assessment

Further characterization of nalmefene for the treatment of anxiety disorders can be assessed in non-clinical models e.g. models for assessment of acute effect as outlined in Examples 4-6. Nalmefene can be assessed in each model both as the sole active substance as well as in combination with a second compound.

Nalmefene can be administered e.g. in the form of nalmefene hydrochloride dissolved in an appropriate amount of saline and dosed to the animals e.g. by subcutaneous administration. A second compound to be combined with nalmefene can be dissolved in an appropriate amount of an appropriate vehicle and dosed to the animals e.g. by subcutaneous administration.

### Example 4

### Vogel Test in Rats

Pairing of water drinking with food shock punishment will induce a conflict in water deprived rats and lead to less frequent water seeking behaviour. Pretreatment of rats with an anxiolytic will counteract the conflict and increase the frequency of water seeking. The test can be conducted as described in detail by Vogel et al., Psychopharmacology, (1971), 21: pp. 1-7. In brief, rats are adapted to the test chambers, a Plexiglas box, equipped with a grid floor made of stainless steel bars and a drinking bottle with tap water. After the adaptation period, the animals are deprived of water and then placed in the test chamber for free access to the drinking bottle for a short period. Afterwards, they are allowed a short free-drinking session in their home cage. After another water deprivation period, the rats are placed again in the test chamber and allowed to drink for a very short time (30 s); immediately afterwards, their drinking attempts are punished with an electric shock. The impulses are delivered every 2 s (timed from the moment when a preceding shock was delivered) between the grid floor and the spout of the drinking bottle. Each shock lasted 1 s; if a rat was drinking when an impulse was released, it received a shock. The number of shocks accepted throughout a 5-min experimental session was recorded.

Nalmefene was tested as the sole active compound in the Vogel model in rats. Rats were deprived of water for approximately 48 hours and were then placed individually into a transparent Plexiglas enclosure (15×32×34 cm) with a floor consisting of stainless steel bars (0.4 cm) spaced 1 cm apart. The rat was left to explore until it found the water spout. Then, every time it drank, it received a slight electric shock (1.7 mA, 1 s) 2 seconds after it started lapping. The number of punished drinks was counted during a 3-minute test. Nalmefene was evaluated at 3 doses (0.01, 0.1 and 1 mg/kg), administered s.c. 30 minutes before the test, and compared with a vehicle control group. No significant effect was shown under the given test conditions.

### Example 5

### Elevated Plus Maze Test in Rats

Rats have an aversion of open spaces and avoid them by confining movements to enclosed spaces or to the edges of a bounded space. Anxiolytic activity can be tested in a test setting consists of a plus-shaped apparatus with two open and two enclosed arms, each with an open roof, elevated 40-70 cm from the floor according to Pellow et al., J. Neurosci. Methods. (1985) August; 14(3):149-67. Pretreatment of rats with anxiolytic active compounds will increase in the proportion of time spent in the open arms (time in open arms/total time in open or closed arms), and an increase in the proportion of entries into the open arms (entries into open arms/total entries into open or closed arms). Total number of arm entries and number of closed-arm entries can be employed as measures of general activity.

Nalmefene was tested as the sole active compound in the elevated plus maze test in rats. A rat was placed in the centre of the plus-maze and left to explore for 5 minutes. The number of entries into the open and closed arms and the time spent on the open arms were recorded. The % of open arm entries (open arm entries/total arm entries×100) was calculated. Nalmefene was evaluated at 3 doses (0.01, 0.1 and 1 mg/kg), administered s.c. 30 minutes before the test, and compared with a vehicle control group. No significant effect was shown at under the given test conditions.

### Example 6

### Marble Burying Test

The method, which detects anxiolytic/tranquillizing activity, follows that described by Broekkamp et al (Eur. J. Pharmacol., 126, 223-229, 1986). Mice exposed to novel objects (marbles) will bury them in the sawdust floor covering. Anxiolytics decrease the number of marbles buried at non-sedative doses.

Nalmefene was tested in the marble burying model as the sole active compound. Mice (NMRI) were individually placed in transparent plastic cages (33×21×18 cm) with 5 cm of sawdust on the floor and 25 marbles grouped in the centre of the cage. The cage was covered with an inverted plastic cage. Each test cage, together with the marbles, was impregnated with mouse odor before-hand by leaving 10 mice in the cage for 15 minutes. These mice then played no further role in the experiment.

The number of marbles covered by sawdust (⅔ or more) was counted at the end of a 30 minute test. 12 mice were studied per group. The test was performed blind. Nalmefene was evaluated at 3 doses (0.01, 0.1 and 1 mg/kg), administered s.c. 30 minutes before the test, and compared with a vehicle control group. Clobazam (8 mg/kg s.c.), administered under the same experimental conditions, was used as reference substance. The experiment therefore included 8 groups.

Data with the test substance were analysed by comparing treated groups with vehicle control using Kruskall-Wallis test followed by Mann-Whitney U tests. Data with the reference substance were analysed using Mann-Whitney U tests. Due to the number of groups, the study was conducted over 2 separate sub-experiments, each sub-experiment including 6 mice per group.

The data showed that nalmefene (0.1 mg/kg), administered s.c. 30 minutes before the test, significantly decreased the number of marbles covered by sawdust, as compared with vehicle controls (−13%, p<0.01). It had no significant effects at 0.01 or 1 mg/kg. Data are further illustrated in Table 9 below.

TABLE 9
Marble burying test in mouse

| | Number of marbles covered by sawdust | |
|---|---|---|
| Treatment | Mean ± SEM | % compared with vehicle |
| Vehicle | 24.8 ± 0.1 | — |
| Nalmefene 0.01 mg/kg | 23.5 ± 1.2 | −5% |
| Nalmefene 0.1 mg/kg | 21.5 ± 1.2 | −13% |
| Nalmefene 1 mg/kg | 22.4 ± 1.4 | −10% |

1-14. (canceled)

15. A method of treating an anxiety disorder comprising administering a therapeutically effective amount of Nalmefene to a patient in need thereof.

16. A method of treating an anxiety disorder comprising administering a therapeutically effective amount of Nalmefene to a patient in need thereof, wherein the patient has alcohol dependence.

17. A method of treating alcohol dependence comprising administering a therapeutically effective amount of Nalmefene to a patient in need thereof, wherein the patient also has a co-morbid anxiety disorder.

18. A method of reducing alcohol consumption and treating an anxiety disorder in a patient comprising administering a therapeutically effective amount of Nalmefene to a patient in need thereof, wherein the patient has alcohol dependence and a co-morbid anxiety disorder.

19. The method of claim 15, wherein said anxiety disorder is an alcohol induced anxiety disorder.

20. The method of claim 17, wherein said co-morbid anxiety disorder is an alcohol induced anxiety disorder.

21. The method of claim 18, wherein said co-morbid anxiety disorder is an alcohol induced anxiety disorder.

22. The method of claim 17, wherein said alcohol dependence is caused by said co-morbid anxiety disorder.

23. The method of claim 18, wherein said alcohol dependence is caused by said anxiety disorder.

24. The method of claim 17, wherein said alcohol dependence and said comorbid anxiety disorder are not causally related to each other.

25. The method of claim 18, wherein said alcohol dependence and said comorbid anxiety disorder are not causally related to each other.

26. The method of claim 15, wherein said anxiety disorder is selected from Acute Stress Disorder, Agoraphobia, Anxiety, Anxiety Disorder, Emotional Distress, Generalised Anxiety Disorder, Nervousness, Nosophobia, Obsessive-Compulsive Disorder, Panic Attack, Panic Disorder, Panic Disorder With Agoraphobia, Post-Traumatic Stress Disorder, Social Phobia, Stress, and Tension.

27. The method of claim 17, wherein said co-morbid anxiety disorder is selected from Acute Stress Disorder, Agoraphobia, Anxiety, Anxiety Disorder, Emotional Distress, Generalised Anxiety Disorder, Nervousness, Nosophobia, Obsessive-Compulsive Disorder, Panic Attack, Panic Disorder, Panic Disorder With Agoraphobia, Post-Traumatic Stress Disorder, Social Phobia, Stress, and Tension.

28. The method of claim 18, wherein said co-morbid anxiety disorder is selected from Acute Stress Disorder, Agoraphobia, Anxiety, Anxiety Disorder, Emotional Distress, Generalised Anxiety Disorder, Nervousness, Nosophobia, Obsessive-Compulsive Disorder, Panic Attack, Panic Disorder, Panic Disorder With Agoraphobia, Post-Traumatic Stress Disorder, Social Phobia, Stress, and Tension.

29. The method of claim 15, wherein nalmefene is the sole active ingredient used in the treatment of said anxiety disorder.

30. The method of claim 17, wherein nalmefene is the sole active ingredient used in the treatment of said anxiety disorder.

31. The method of claim 15, wherein said patient is further treated with a second compound which is an antianxiety agent.

32. The method of claim 17, wherein said patient is further treated with a second compound which is an antianxiety agent.

33. The method of claim 18, wherein said patient is further treated with a second compound which is an antianxiety agent.

34. The method of claim 15, wherein the therapeutically effective amount of nalmefene is between 10 and 20 mg.

35. The method of claim 18, wherein the therapeutically effective amount of nalmefene is between 10 and 20 mg.
